(19) United States
(12) Patent Application Publication
(10) Pub. No.: US 2006/0135854 A9
(43) Pub. Date: Jun. 22, 2006

(54) Sleep study software architecture

(75) Inventors: Sean P. McDonough, Roswell, GA (US); Herb Stevenson, Tampa, FL (US); Kevin Smith, Boston, GA (US)

Correspondence Address: SHOEMAKER AND MATTARE, LTD, 10 POST OFFICE ROAD - SUITE 110, SILVER SPRING, MD, 20910, US

(73) Assignee: SleepEx Systems, Inc., Marietta, GA (US)

(21) Appl. No.: 10/439,305

(22) Filed: May 16, 2003

Related U.S. Application Data
(60) Provisional application No. 60/378,122, filed on May 16, 2002.

(51) Int. Cl. A61B 5/00

(52) U.S. Cl. 600/300; 709/213

(57) ABSTRACT

Sleep study test results and patient information gathered at remote test sites are compressed, encrypted, and transmitted to a central server for storage. A test scoring technician, at a remote site may, upon proper authentication, access the data, interpret it, and generate and store his interpretation. A physician at another remote site may also, upon proper authentication, access the data and the scorer's interpretation thereof.

### BACKGROUND OF THE INVENTION

This invention is directed to software specifically designed for sleep studies.

The software resulted from a desire to combine the output generated by Respironic's Alice software and other acquisition equipment in the industry, which produces graphs and reports based on data from a polysomnogram, with data that has been manually entered either online or on paper reports. This data was previously transferred via fax, file transfer, and overnight delivery between sleep labs, scoring centers, and evaluating physicians.

### SUMMARY OF THE INVENTION

An object of the invention is to automate and streamline the business processes of administering, scoring, and interpreting sleep studies conducted by hospital and physician owned sleep diagnostic facilities.

These and other objects are attained by the sleep study software architecture described below.

### BRIEF DESCRIPTION OF THE DRAWINGS

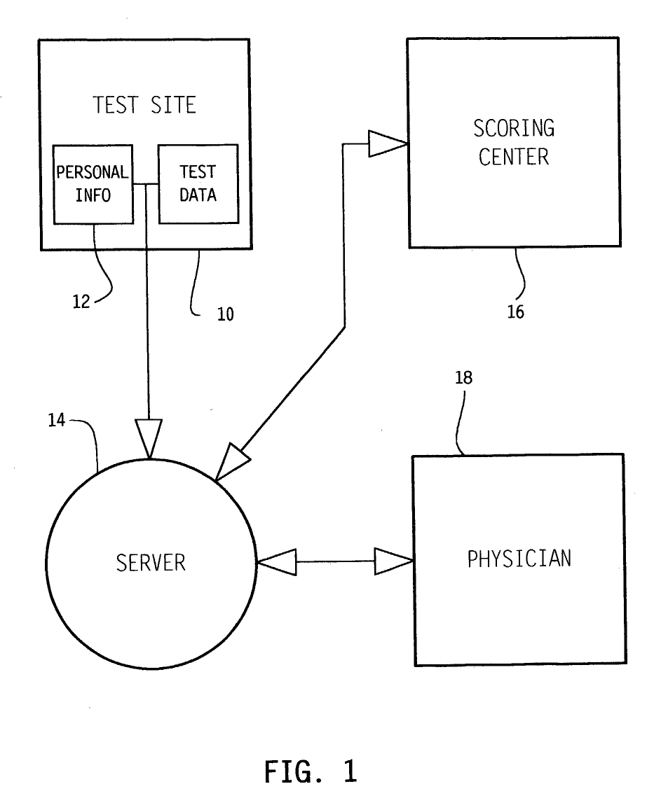

In the accompanying drawings, FIG. 1 is a schematic diagram of sleep study software architecture embodying the invention.

### DESCRIPTION OF THE PREFERRED EMBODIMENT

The method of this invention includes three distinct processes which, together, create an end-to-end solution. The software implementing the method was written in Visual Basic 6.0, utilizing Virtual Private Networking (VPN) for secure transmissions of tests to the central server, an encrypted file compression utility to more efficiently send files to store individual patient records for the software, file transfer protocol (FTP) via the Microsoft Windows API, to send smaller packets of information, and a system specific Internet web browser for scoring tests and viewing patient information on a central server.

As shown in FIG. 1, the invention provides the administering technician at a remote testing facility 10 with five screens for data input while conducting a sleep study. The first screen is for general patient information 12 including name, height, weight, etc. The second screen is for additional patient information, which include a list of thirty-five questions that are asked of the patient to better understand their condition. The third screen is a technical observations screen, which allows the technician to input data about the patient while the test is ongoing. The fourth screen is a polysomnogram comments section, which allows the technician to input additional information about the polysomnograph while the test is occurring. The fifth screen is the technical assessment form, which allows the technician to summarize the test results before sending the test to be scored and evaluated. Once this test has been transmitted, this information may not be changed. The information contained in the technical observations, polysomnogram comments, and technical assessment forms may be merged into printable document templates which can then be printed, faxed, and stored, if necessary.

At the test's conclusion and after all data has been entered into the system, the product transmits the information to the central server 14 where it is stored in a location specific to the attending sleep lab. This process includes automatically detecting the last test finished by the acquisition equipment, creating a patient information file, compressing the test results and patient information file, establishing a secure VPN connection to the central server, mapping a virtual drive to the central server, and sending the data to the central server.

After all data has been sent, a request is sent to the web service, located on the central server, to decompress the files so that they will be ready for viewing by a representative of the scoring center.

The scoring center 16 is manually notified that a test has been completed and logs on to the system to score the test. The invention has three modes that the scoring center may use to score a test: online, download, and offline.

In online mode, the system connects to the central server and displays a list of all sites that are serviced by this program. Once a site is selected, a list of patients associated with that site is presented to the user for selection. A request is then sent to the web service to prepare the server for the incoming user. This service ensures that Alice's data and report directory settings are pointing to the correct place, and that the user is authorized to view the patient information that he has requested. After a site and patient have been selected, the system Web Browser is activated, which provides a “window” to the central server. Once this connection has been established, the scorer is re-authenticated on the server and asked to provide his user name one more time for security purposes. The browser displays a remote version of the system for viewing of patient information and an instance of the Alice software for scoring the test.

The scoring technician uses Alice or other interface software to score the test and generates customized technical reports based on the interpretation of the test by Alice. The technician edits the test results as needed and saves the reports, in rich text format, to a pre-defined directory on the server. The invention monitors this pre-defined directory so that it may link the patient records to the correct reports for viewing by a doctor.

Download mode is similar to the online process, but it uses FTP to download the compressed test results and patient information file to the user's computer after a sleep clinic name and patient have been selected. After the files have been downloaded they are decompressed and placed in Alice's data directory and the software starts Alice. The scoring technician may then score the test using the Alice software, and will then, once again, save the tests to a pre-defined location. After the reports have been created, the technician clicks a button in the software to associate the respective reports with the patient and transmit the reports back to the central server. It is not necessary to transmit the entire test back to the server, because no changes have been made to the original data that was downloaded from the server.

Offline mode provides a mechanism for the technician to view and score a test after a test has been downloaded, but with no Internet connection to send the reports back to the central server. The technician may transmit reports once an Internet connection has been re-established.

After a test has been conducted and scored, it is sent to a physician, at another remote site 18, to be evaluated. The physician, running the software, is presented with two options when opening a patient record: he may download the test, or view it online.

In online mode, the system connects to the central server and displays a list of all sites that are serviced by the program. Once a site is selected, a list of patients associated with that site is presented to the doctor for selection. A request is then sent to the web service to prepare the server for the incoming user. This service ensures that Alice's data and report directory settings are pointing to the correct place, and that the user is authorized to view the patient information that they have requested. Physicians may only look at patient information for the sites that they have been give specific authorization for the system administrators and support staff. After a site and patient have been selected the system Web Browser is generated, which provides the doctor a “window” to the central server. The doctor is asked to re-authenticate his/her user name and password. Upon completion of all authorizations, the software and Alice are started automatically on the server. Alice is pointed to the specific tests that the doctor is looking for and the system displays an option to view the study or view the interpretative reports created for the physician. The doctor then may evaluate the test results, make modifications to the reports, if necessary, and save the reports back to the pre-defined location.

Download mode is similar to online mode, where the doctor is asked to select a site and patient to evaluate. Once all authentications are completed, the test, scoring reports, and patient information files are downloaded to the doctor's computer. Alice is started and the report documents are opened for the doctor. The doctor may then view the results and make adjustments to the reports, if necessary. After all is completed the doctor clicks a button and the reports are transmitted back to the server.

Also included with the product is an self-checking product update feature. When the application starts it first checks the central server for new versions of the software. If the application finds a newer version on the central server, the new version is downloaded to the end-user's computer. Once downloaded, a file copy program moves the new version into the program home directory.

Another piece of the architecture is the system Web Service. This application resides on the central server and is responsible for an additional level of user authentication, to prepare the server for when a user is about to login to the server, and to decompress tests sent to the central server by the sleep labs.

The first task of the web service is to decompress the files sent to the central server by the sleep lab. Once the sleep lab has finished its transmission, a message is sent to the web service containing the patient name and site name pertaining to the files sent to the server. The web service then decompresses these files into the correct site folder on the server. This ensures that the VPN connection is established for the least amount of time and ensures proper decompression of the files since the decompression process can not be interrupted by network connection errors.

Secondly, when a user attempts to log in to the server, a message is sent from the user's computer to the system Web Service containing the user name attempting to connect, as well as the site and patient that he is interested in viewing. The web service receives the message, verifies the user's authority level against an administered database, and either allows or denies the user access to the patient list and/or server.

Lastly, once a user is successfully validated, the web service adjusts the settings of the server application and the Alice registry settings in preparation for the incoming user. These adjustments ensure that Alice and the system are looking in the correct location on the server for the patient and Alice test files.

The system uses a file organization structure to contain all of the information regarding the patient. This information is stored in files that end with a .pat file extension and are stored in the Patients sub-directory of the installation directory. This file is passed back and forth to each machine that makes adjustments or needs to view the patient information file. This file is passed between computers so that patient information can be viewed “offline”, meaning no Internet connection is required to view the patient file and score a test.

The Alice data is retrieved by reading the Alice registry entry pertaining to the location of test results, compressing the folders, and transferring the data to the central server into a folder encapsulating all patient information files and Alice test results for a specific site.

Since the invention is subject to modifications and variations, it is intended that the foregoing description and the accompanying drawings shall be interpreted as only illustrative of the invention defined by the following claims.

We claim:

1. A method of storing patient information and sleep test data on a central server, said method comprising steps of
obtaining information about a patient and storing said information at at least one remote site,
testing the patient for sleep activity at said remote site and producing test data,
combining and transmitting said information and said test data from said remote site to said central server,
making said combined information and test data available to a test scorer at another remote site and allowing the scorer to score the test data and generate an interpretation of the combined information and test data, and
making said combined information, test data, and interpretation, if generated, available to a physician at another remote site.

2. The invention of claim 1, further comprising a step of compressing and encrypting said patient information and test data prior to said transmitting step.

3. The invention of claim 1, comprising a further step of authenticating said scorer before the step of making the combined information and test data available to the scorer.

4. The invention of claim 1, comprising a further step of authenticating said physician before the step of making the combined information, test data and interpretation available to the physician.

5. The invention of claim 1, further comprising a step of notifying said physician when said information and test data are available for access.

6. A sleep study information gathering and dissemination system comprising
a central server,
a plurality of remote test stations,
means for encrypting and compressing test results and patient information gathered at said remote test stations to said central server,
means for making said test results and patient information available to a test scorer at a remote location upon proper authentication, and
means for making said test results and patient information, and any interpretation generated by said scorer, to a physician at a remote location upon proper authentication.
